(19) United States
(12) Patent Application Publication
(10) Pub. No.: US 2023/0230703 A1
(43) Pub. Date: Jul. 20, 2023

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Yudai YAMAZAKI, Nasushiobara (JP); Sho SASAKI, Utsunomiya (JP); Kosuke ARITA, Otawara (JP); Longxun PIAO, Nasushiobara (JP)

(73) Assignee: CANON MEDICAL SYSTEMS CORPORATION, Otawara-shi (JP)

(21) Appl. No.: 18/147,333

(22) Filed: Dec. 28, 2022

(30) Foreign Application Priority Data
Jan. 18, 2022 (JP) 2022-005850

(51) Int. Cl. G16H 50/20

(52) U.S. Cl. CPC G16H 50/20; G16H 50/30

(57) ABSTRACT

A medical information processing device of an embodiment includes a processing circuitry. The processing circuitry extracts first medical treatment information related to diagnostic support information from among a plurality of pieces of medical treatment information on the basis of a corresponding relationship in which the diagnostic support information and the plurality of pieces of medical treatment information are associated, and a degree of contribution of each of the plurality of pieces of medical treatment information to the diagnostic support information, extracts second medical treatment information related to the first medical treatment information from among the plurality of pieces of medical treatment information on the basis of degrees of mutual relevance of the medical treatment information and the degree of contribution of each of the plurality of pieces of medical treatment information, and causes a display unit to display these information.

### CROSS-REFERENCE TO RELATED APPLICATION

The present application claims priority based on Japanese Patent Applications No. 2022-005850 filed Jan. 18, 2022, the content of which is incorporated herein by reference.

### FIELD

Embodiments disclosed in the present specification and drawings relate to a medical information processing device, a medical information processing method, and a storage medium.

### BACKGROUND

Since cancer treatment is cardiotoxic, it is important to ascertain a risk of heart disease of a patient before treatment. Therefore, Current Decision Support (CDS) has been developed. In CDS, it is important to show the grounds for estimation results, and doctors can consider a treatment policy by ascertaining the grounds. Recurrent Attentive and Intensive Model (RAIM) can calculate a disease risk (support information) from a large number of examination values (medical information) using a machine learning model and the like and extract examination items that have contributed to calculation of the disease risk from the large number of examination values. Medically unknown medical treatment information that is not related to a disease may be used as an input, and the model may extract medically unknown medical treatment information as grounds. When medically unknown medical treatment information is presented, a user may not understand why it has been extracted as grounds.

In regards to this, for example, technology for generating graphs representing risks, topics (words) that are factors of the risks, and relationships on the basis of medical documents and academic papers is known. Conventional technology generates graphs on the basis of medically known data such as medical documents and academic papers, and thus does not cover medically unknown medical treatment information and cannot present the relevance to users.

### BRIEF DESCRIPTION OF THE DRAWINGS

FIG. 1 is a diagram showing an example of a configuration of a medical information processing device in an embodiment.

FIG. 2 is a flowchart showing a flow of a series of processing of a processing circuitry according to an embodiment.

FIG. 3 is a diagram showing an example of a degree of contribution of each piece of medical treatment information output by a machine learning model.

FIG. 4 is a diagram showing an example of a medical relevance database.

FIG. 5 is a diagram showing an example of weights (degrees of relevance) between a plurality of pieces of medical treatment information output by a machine learning model.

FIG. 6 is a diagram showing an example of content to be displayed on a display.

FIG. 7 is a diagram showing another example of content to be displayed on the display.

FIG. 8 is a diagram showing another example of content to be displayed on the display.

FIG. 9 is a diagram showing another example of content to be displayed on the display.

### DETAILED DESCRIPTION

Hereinafter, a medical information processing device, a medical information processing method, and a storage medium according to embodiments will be described with reference to the drawings.

A medical information processing device of an embodiment is a medical information processing device for supporting interpretation of output results of a model that outputs diagnostic support information which is information for supporting diagnosis of a subject in response to input of a plurality of pieces of medical treatment information obtained at the time of medical treatment of the subject. The medical information processing device includes a first extraction unit, a second extraction unit, and a display control unit. The first extraction unit extracts first medical treatment information related to the diagnostic support information from among the plurality of pieces of medical treatment information on the basis of a corresponding relationship in which the diagnostic support information and the plurality of pieces of medical treatment information are associated, and a degree of contribution of each of the plurality of pieces of medical treatment information input to the model at the time of causing the model to output the diagnostic support information to the diagnostic support information. The second extraction unit extracts second medical treatment information related to the first medical treatment information from among the plurality of pieces of medical treatment information on the basis of degrees of mutual relevance of the medical treatment information input to the model at the time of causing the model to output the diagnostic support information and the degree of contribution of each of the plurality of pieces of medical treatment information. The display control unit causes a display unit to display the diagnostic support information, the first medical treatment information, and the second medical treatment information. As a result, it is possible to allow a user to better understand output results of a machine learning model that outputs diagnostic support information that is information for supporting diagnosis of a subject in response to input of a plurality of pieces of medical treatment information obtained at the time of medical treatment of the subject.

### [Configuration of Medical Information Processing Device]

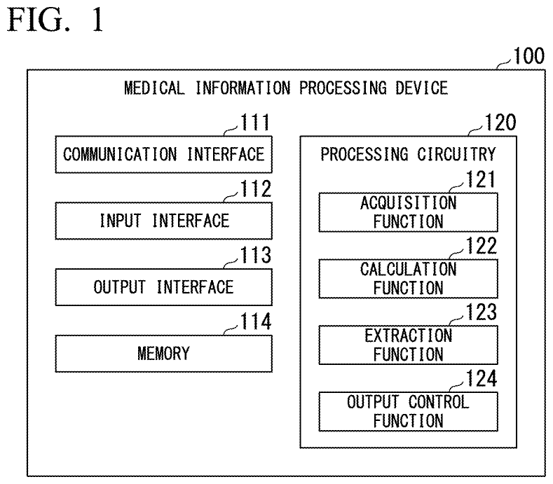

FIG. 1 is a diagram showing an example of a configuration of a medical information processing device 100 in an embodiment. The medical information processing device 100 includes, for example, a communication interface 111, an input interface 112, an output interface 113, a memory 114, and a processing circuitry 120.

The communication interface 111 communicates with external devices via a communication network NW. The communication network NW may refer to general information communication networks using telecommunication technology. For example, the communication network NW includes a wireless/wired local area network (LAN) such as a hospital backbone LAN, the Internet, a telephone communication network, an optical fiber communication network, a cable communication network, a satellite communication network, and the like. The communication interface 111 includes, for example, a network interface card (NIC), an antenna for wireless communication, and the like.

The input interface 112 receives various input operations from an operator, converts the received input operations into electrical signals, and outputs the electrical signals to the processing circuitry 120. For example, the input interface 112 includes a mouse, a keyboard, a trackball, a switch, a button, a joystick, a touch panel, and the like. The input interface 112 may be, for example, a user interface that receives audio input, such as a microphone. When the input interface 112 is a touch panel, the input interface 112 may also have a display function of a display 113a included in the output interface 113, which will be described later.

It should be noted that the input interface 112 in this specification is not limited to one including physical operation components such as a mouse and a keyboard. For example, examples of the input interface 112 also include an electrical signal processing circuitry that receives an electrical signal corresponding to an input operation from an external input apparatus provided separately from the device and outputs the electrical signal to a control circuit.

The output interface 113 includes, for example, the display 113a, a speaker 113b, and the like. The display 113a displays various types of information. For example, the display 113a displays an image generated by the processing circuitry 120, a graphical user interface (GUI) for receiving various input operations from the operator, and the like. For example, the display 113a is a liquid crystal display (LCD), a cathode ray tube (CRT) display, an organic electroluminescence (EL) display, or the like. The speaker 113b outputs information input from the processing circuitry 120 as sound.

The memory 114 is realized by, for example, a random access memory (RAM), a semiconductor memory device such as a flash memory, a hard disk, or an optical disc. Such non-transient storage media may be realized by other storage devices such as a network attached storage (NAS) and an external storage server devices connected via the communication network NW. Further, the memory 114 may also include non-transient storage media such as a read only memory (ROM) and a register.

The memory 114 stores model information in addition to programs executed by a hardware processor. The model information is information (program or algorithm) that defines a machine learning model 200 trained to output at least diagnostic support information that is information for supporting diagnosis of a subject (for example, a human patient) in response to input of a plurality of pieces of medical treatment information obtained at the time of medical treatment of the subject.

The machine learning model 200 may be implemented by, for example, a neural network. More specifically, the machine learning model 200 may be implemented by a convolutional neural network having an attention mechanism. Further, the machine learning model 200 is not limited to neural networks and may be implemented by other models such as support vector machines, decision trees, naive Bayes classifiers, and random forests.

When the machine learning model 200 is implemented by a neural network, the model information includes, for example, coupling information representing how units included in each of an input layer, one or more hidden layers (intermediate layers), and an output layer that constitute the neural network are coupled to each other, weight information representing how many coupling coefficients are provided to data input/output between coupled units, and the like. The coupling information includes, for example, information such as the number of units included in each layer, information for designating the type of a unit that is a coupling destination of each unit, an activation function that realizes each unit, and a gate provided between units in the hidden layers. The activation function that realizes a unit may be, for example, a rectified linear unit (ReLU) function, an exponential linear units (ELU) function, a clipping function, a sigmoid function, a step function, a hyperbolic tangent function, an identity function, or the like. The gate selectively passes or weights data transmitted between units, for example, depending on a value (e.g., 1 or 0) returned by the activation function. A coupling coefficient includes, for example, a weight applied to output data when data is output from a unit in a certain layer to a unit in a deeper layer in a hidden layer of the neural network. Further, the coupling coefficients may also include a bias component unique to each layer, and the like.

The memory 114 also stores a medical relevance database in addition to a program and model information. The medical relevance database is, for example, a database in which the presence or absence of medical relevance of parameters such as a blood pressure and a heart rate is associated with diseases such as heart diseases and cancers. The medical relevance database is an example of a “corresponding relationship.”

The processing circuitry 120 includes, for example, an acquisition function 121, a calculation function 122, an extraction function 123, and an output control function 124. The processing circuitry 120 realizes these functions by, for example, a hardware processor (computer) executing a program stored in the memory 114 (storage circuit). The extraction function 123 is an example of a “first extraction unit,” a “second extraction unit,” and a “third extraction unit.” The output control function 124 is an example of a “display control unit.”

The hardware processor in the processing circuitry 120 refers to, for example, a circuit (circuitry) such as a central processing unit (CPU), a graphics processing unit (GPU), an application specific integrated circuit (ASIC), and a programmable logic device (for example, a simple programmable logic device (SPLD), a complex programmable logic device (CPLD), or a field programmable gate array (FPGA)). Instead of storing the program in the memory 114, the program may be directly incorporated into the circuit of the hardware processor. In this case, the hardware processor realizes the functions by reading and executing the program incorporated into the circuit. The aforementioned program may be stored in the memory 114 in advance, or may be stored in a non-transient storage medium such as a DVD or a CD-ROM and installed in the memory 114 from the non-transient storage medium by setting the non-transient storage medium in a drive device (not shown) of the user interface 10. The hardware processor is not limited to being configured as a single circuit, and may be configured as one hardware processor by combining a plurality of independent circuits to realize each function. Further, a plurality of components may be integrated into one hardware processor to realize each function.

### [Processing Flow of Medical Information Processing Device]

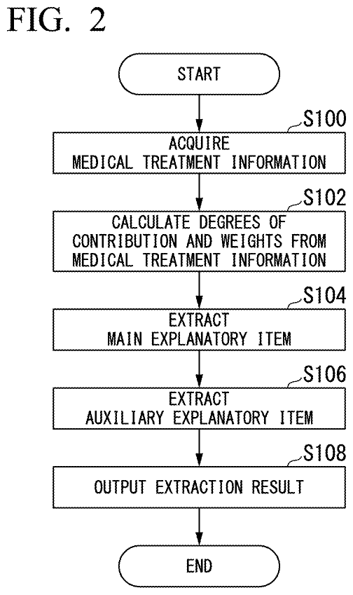

Hereinafter, a series of processing performed by the processing circuitry 120 of the medical information processing device 100 will be described below with reference to a flowchart. FIG. 2 is a flowchart showing a flow of a series of processing of the processing circuitry 120 according to an embodiment.

First, the acquisition function 121 acquires a plurality of pieces of medical treatment information obtained at the time of medical treatment of a patient that is a diagnosis target (step S100). The medical treatment information includes, for example, parameters (examination items) such as a blood pressure, a heart rate, SpO2 (percutaneous arterial blood oxygen saturation), a body weight, a blood sugar level, NT-proBNP, and cardiac troponin I.

For example, it is assumed that a medical staff member such as a doctor or a nurse inputs a plurality of parameters such as the blood pressure and heart rate of a patient to the user interface 10. In this case, the acquisition function 121 acquires each parameter input to the user interface 10 as medical treatment information. Further, when the medical staff member inputs a plurality of parameters of the patient to a dedicated terminal in the hospital instead of the user interface 10, for example, the acquisition function 121 may communicate with the dedicated terminal via the communication interface 111 to acquire each input parameter input to the dedicated terminal as medical treatment information.

Next, when the acquisition function 121 acquires a plurality of pieces of medical treatment information (a plurality of parameters such as a blood pressure and a heart rate), the calculation function 122 calculates a degree of contribution of each piece of medical treatment information and weights (also referred to as degrees of relevance) between the plurality of pieces of medical treatment information from the plurality of pieces of medical treatment information acquired by the acquisition function 121 using the machine learning model 200 defined by model information stored in the memory 114 (step S102).

As described above, when a plurality of pieces of medical treatment information is input to the machine learning model 200, the machine learning model 200 outputs diagnostic support information.

Diagnostic support information is, for example, information representing, as a quantitative risk, a probability of a patient having already suffered from a certain target disease (heart disease, cancer, or the like) at the time of medical treatment of the patient (at the time when the patient was medically treated to obtain medical treatment information). Further, the diagnostic support information may be, for example, information representing, as a quantitative risk, a probability of a patient suffering from a certain target disease at a certain time in the future from a medical treatment time of the patient. That is, the machine learning model 200 outputs the probability of the patient having already suffered from the target disease or suffering from it in the future as diagnostic support information.

The machine learning model 200 is typically trained using, as training data, a data set in which a disease that a certain learning subject has already suffered from or a disease that is highly likely to occur in the future is labeled for a plurality of pieces of medical treatment information derived from the learning subject. In other words, the machine learning model 200 is a model trained to output a disease that a certain learning subject has already suffered from or a disease that is highly likely to occur in the future when a plurality of pieces of medical treatment information derived from the learning subject is input thereto. A learning subject may be a patient who has been a diagnosis target in the past. That is, a learning subject may be the same person as a diagnosis subject, or may be a different person.

The machine learning model 200 trained in this manner outputs diagnostic support information (disease risk) as an estimation result when a plurality of pieces of diagnostic information of a certain diagnosis subject is input thereto (machine learning model 200). The estimation result of the machine learning model 200 is represented by, for example, a multidimensional vector or tensor. The vector or tensor contains a probability of having a disease as an element value. For example, it is assumed that there are a total of three types of diseases, disease A, disease B, and disease C, as diseases that a diagnosis subject may suffer from. In this case, a vector or a tensor can be represented as (e1, e2, e3) where e1 is the probability of disease A, e2 is the probability of disease B, and e3 is the probability of disease C.

Furthermore, the machine learning model 200 simultaneously outputs a degree of contribution of each piece of medical treatment information and weights (degrees of relevance) between a plurality of pieces of medical treatment information in addition to the diagnostic support information (disease risk).

A degree of contribution of medical treatment information is an index indicating a degree to which each piece of medical treatment information input to the machine learning model 200 contributes to diagnostic support information that is an output result of the machine learning model 200 when the machine learning model 200 is caused to output the diagnostic support information (disease risk).

A weight (degree of relevance) between medical treatment information is an index indicating a degree to which certain medical treatment information input to the machine learning model 200 is related to all other medical treatment information that are also input to the machine learning model 200 when the machine learning model 200 is caused to output the diagnostic support information (disease risk).

For example, when the machine learning model 200 is implemented by a convolutional neural network having an attention mechanism, the attention mechanism calculates a degree of contribution of each piece of medical treatment information to diagnostic support information and weights (degrees of relevance) between a plurality of pieces of medical treatment information.

In addition, the calculation function 122 may calculate a degree of contribution of each piece of medical treatment information to diagnostic support information and weights (degrees of relevance) between a plurality of pieces of medical treatment information using a visualization method called class activation mapping (CAM). For example, gradient-weighted class activation mapping (Grad-CAM) or the like can be used as a class activation mapping technique.

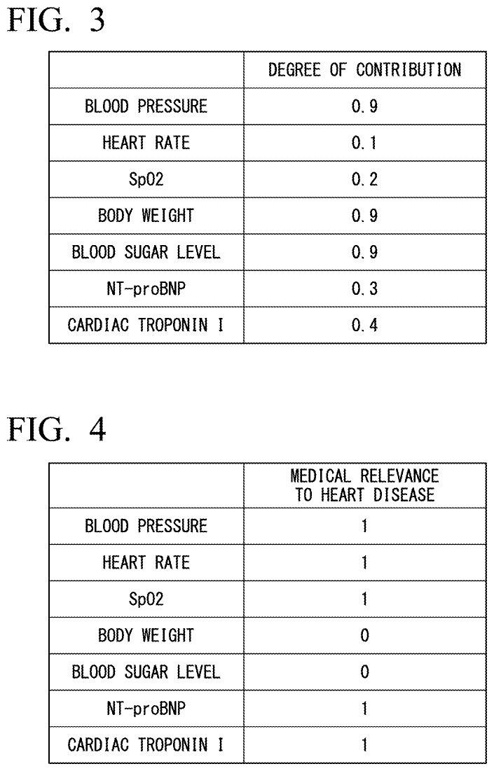

FIG. 3 is a diagram showing an example of a degree of contribution of each piece of medical treatment information output by the machine learning model 200. As illustrated, the machine learning model 200 calculates degrees of contribution of a plurality of pieces of medical treatment information (parameters) such as a blood pressure, a heart rate, SpO2, a body weight, a blood sugar level, NT-proBNP, and cardiac troponin I to diagnostic support information (disease risk).

Return to description of the flowchart of FIG. 2. Subsequently, the extraction function 123 extracts medical treatment information (hereinafter referred to as main explanatory items) related to the diagnostic support information (disease risk) from a plurality of pieces of medical treatment information input to the machine learning model 200 on the basis of the medical relevance database stored in the memory 114 and the degree of contribution of each piece of medical treatment information calculated by the calculation function 122 (step S104). The main explanatory item is an example of “first medical treatment information.”

FIG. 4 is a diagram showing an example of the medical relevance database. As illustrated, the medical relevance database is table data in which the presence or absence of medical relevance of each piece of medical treatment information (parameter) is associated as a numerical value such as “1” or “0” with a certain target disease such as a heart disease. The medical relevance database in the illustrated example represents that, among seven types of medical treatment information (parameters), namely a blood pressure, a heart rate, SpO2, a body weight, a blood sugar level, NT-proBNP, and cardiac troponin I, five types of medical treatment information, namely the blood pressure, the heart rate, SpO2, proBNP, and cardiac troponin I, have medical relevance to the heart disease (a table field is “1”), and two types of medical treatment information, namely the body weight and the blood sugar level, have no medical relevance to the heart disease (table field is “0”).

For example, it is assumed that seven types of medical treatment information, namely the blood pressure, heart rate, SpO2, body weight, blood sugar level, NT-proBNP, and cardiac troponin I, are input to the machine learning model 200, and the machine learning model 200 outputs a risk of heart disease as diagnostic support information. In this case, first, the extraction function 123 extracts medical treatment information having medical relevance to the heart disease (having a field of “1”) from the seven types of medical treatment information input to the machine learning model 200 with reference to the medical relevance database. In the example of FIG. 4, five types of medical treatment information are extracted.

Furthermore, the extraction function 123 extracts, as main explanatory items, a predetermined number of pieces of high-ranking medical treatment information having high degrees of contribution to the diagnostic support information regarding the heart disease from among the five types of medical treatment information having medical relevance to the heart disease while referring to a table of degrees of contribution of medical treatment information, as illustrated in FIG. 3. The predetermined number is a natural number (a non-zero positive integer). For example, if the predetermined number is 1, the blood pressure having the highest degree of contribution (degree of contribution of 0.9) among the five types of medical treatment information is extracted as a main explanatory item.

In addition, the extraction function 123 may extract, as main explanatory items, all medical treatment information whose degrees of contribution to the diagnostic support information regarding the heart disease are equal to or greater than a threshold value from among the five types of medical treatment information having medical relevance to the heart disease. For example, if the threshold value is 0.4, the blood pressure having a degree of contribution of 0.9 and cardiac troponin I having a degree of contribution of 0.4 are extracted as main explanatory items from among the five types of medical treatment information.

Return to description of the flowchart of FIG. 2. Subsequently, the extraction function 123 extracts medical treatment information (hereinafter referred to as auxiliary explanatory items) related to the main explanatory items from among the plurality of pieces of medical treatment information input to the machine learning model 200 on the basis of the degree of contribution of each piece of medical treatment information and the weights (degrees of relevance) between the plurality of pieces of medical treatment information calculated by the calculation function 122 (step S106). The auxiliary explanatory item is an example of “second medical treatment information.”

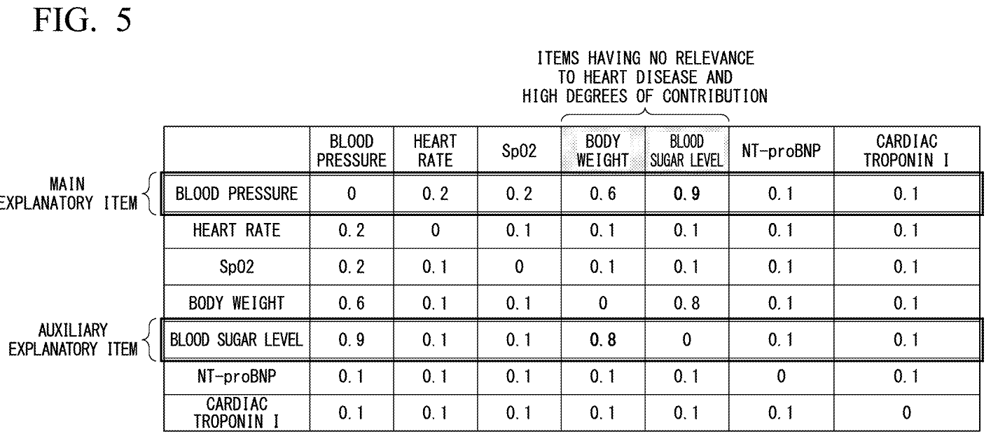

FIG. 5 is a diagram showing an example of weights (degrees of relevance) between a plurality of pieces of medical treatment information output by the machine learning model 200. As illustrated, for each piece of medical treatment information, weights (degrees of relevance) with respect to all other pieces of medical treatment information are calculated. For example, it is assumed that the blood pressure is extracted as a main explanatory item for diagnostic support information regarding the heart disease from among seven types of medical treatment information input to the machine learning model 200. In this case, the extraction function 123 extracts medical treatment information having no medical relevance to the heart disease as candidates for auxiliary explanatory items with reference to the topmost record in the table of FIG. 5 that stores weights of other pieces of medical treatment information with respect to the blood pressure. In the illustrated example, the body weight and the blood sugar level are extracted as candidates for auxiliary explanatory items.

When the extraction function 123 extracts one or a plurality of candidates for auxiliary explanatory items from among the seven types of medical treatment information input to the machine learning model 200, the extraction function 123 extracts, as auxiliary explanatory items, a predetermined number of high-ranking candidates having high weights (degrees of relevance) with respect to the main explanatory items from among the one or plurality of candidates for auxiliary explanatory items. For example, if the predetermined number is 1, the blood sugar level having a higher weight with respect to the blood pressure that is the main explanatory item is extracted as a formal auxiliary explanatory item from the body weight and the blood sugar level that are candidates for auxiliary explanatory items.

In addition, when the extraction function 123 extracts one or a plurality of candidates for auxiliary explanatory items from seven types of medical treatment information input to the machine learning model 200, the extraction function 123 may extract, as auxiliary explanatory items, all candidates whose weights (degrees of relevance) with respect to the main explanatory item are equal to or greater than a threshold value from the one or plurality of candidates for auxiliary explanatory items. For example, if the threshold value is 0.5, the body weight and the blood sugar level are extracted as auxiliary explanatory items because the weight of the body weight that is one of the candidates for auxiliary explanatory items with respect to the blood pressure is 0.6, the weight of the blood sugar level that is another candidate with respect to the blood pressure is 0.9, and both are equal to or greater than the threshold value of 0.5.

Furthermore, when the extraction function 123 extracts one or a plurality of auxiliary explanatory items, the extraction function 123 may extract medical treatment information (hereinafter referred to as a second auxiliary explanatory item) related to the auxiliary explanatory items from the plurality of pieces of medical treatment information input to the machine learning model 200 on the basis of the degree of contribution of each piece of medical treatment information and the weights (degrees of relevance) between the plurality of pieces of medical treatment information. The second auxiliary explanatory item is an example of “third medical treatment information.”

For example, it is assumed that the blood sugar level is extracted as an auxiliary explanatory item. In this case, the extraction function 123 extracts medical treatment information having no medical relevance to the heart disease as candidates for the second auxiliary explanatory item with reference to the record in the fifth row from the top in the table of FIG. 5, which stores weights of other pieces of medical treatment information with respect to the blood sugar level. In the illustrated example, the body weight is extracted as a candidate for the second auxiliary explanatory item (the blood sugar level has already been extracted and thus is not re-extracted).

When the extraction function 123 extracts one or a plurality of candidates for the second auxiliary explanatory item, the extraction function 123 extracts, as candidates for the second auxiliary explanatory item, a predetermined number of high-ranking candidates having high weights (degrees of relevance) with respect to the auxiliary explanatory items from the one or plurality of candidates for the second auxiliary explanatory item. In the illustrated example, there is only one type of candidate for the second auxiliary explanatory item, namely the body weight, and thus the body weight is automatically extracted as the second auxiliary explanatory item.

Further, when the extraction function 123 extracts one or a plurality of candidates for the second auxiliary explanatory item, the extraction function 123 may extract, as the second auxiliary explanatory items, all candidates whose weights (degrees of relevance) with respect to the auxiliary explanatory items are equal to or greater than a threshold value from the one or plurality of candidates for the second auxiliary explanatory item.

Return to the description of the flowchart of FIG. 2. Subsequently, the output control function 124 outputs the extraction result of the extraction function 123 (step S108).

For example, the output control function 124 may cause the display 113a of the output interface 113 to display content including the main explanatory items and the auxiliary explanatory items extracted by the extraction function 123 and the diagnostic support information (disease risk) calculated by the calculation function 122.

Further, the output control function 124 may transmit the content to an external device (e.g., an intra-hospital dedicated terminal used to input medical treatment information) via the communication interface 111 instead of or in addition to causing the display 113a to display the content.

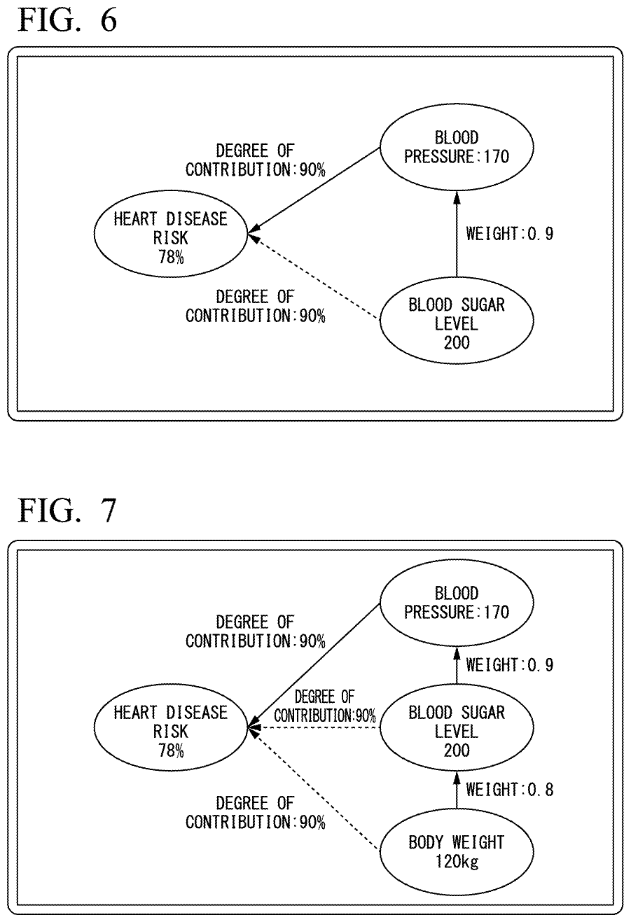

FIG. 6 is a diagram showing an example of content displayed on the display 113a. In the figure, the node indicating a heart disease risk represents a probability calculated by the machine learning model 200 as diagnostic support information, the node indicating a blood pressure represents medical treatment information extracted as a main explanatory item, and the node indicating a blood sugar level represents medical treatment information extracted as an auxiliary explanatory item.

For example, the output control function 124 determines a content display mode on the basis of a degree of contribution of the main explanatory item to the diagnostic support information, a degree of contribution of the auxiliary explanatory item to the diagnostic support information, and the mutual weights (degrees of relevance) of the main explanatory item and the auxiliary explanatory item. Specifically, the output control function 124 displays, as content, a directed graph in which a unidirectional solid-line arrow is provided on the edges between the node of the diagnostic support information and the node of the main explanatory item, a unidirectional dashed-line arrow is provided on the edges between the node of the diagnostic support information and the node of the auxiliary explanatory item, and a unidirectional solid-line arrow is provided on the edges between the node of the main explanatory item and the node of the auxiliary explanatory item while distinguishing the colors and patterns of the nodes. On the directed graph, each edge may be associated with a degree of contribution and a weight (degree of relevance).

Further, when the extraction function 123 has extracted the second auxiliary explanatory item in addition to the auxiliary explanatory item, the output control function 124 may cause the display 113a to display content including the diagnosis support information (disease risk), the main explanatory item, the auxiliary explanatory item, and the second auxiliary explanatory item.

FIG. 7 is a diagram showing another example of content displayed on the display 113a. In the figure, the node indicating a heart disease risk represents a probability calculated by the machine learning model 200 as diagnostic support information, the node indicating a blood pressure represents medical treatment information extracted as a main explanatory item, the node indicating a blood sugar level represents medical treatment information extracted as an auxiliary explanatory item, and the node indicating a body weight represents medical treatment information extracted as a second auxiliary explanatory item. When such a second auxiliary explanatory item is extracted, the output control function 124 may also display a directed graph in which the colors and patterns of the nodes are distinguished as content.

Although the machine learning model 200 mainly outputs only the risk of heart disease as diagnostic support information in the above description, a risk of disease other than the heart disease may be output, for example. In this case, a main explanatory item and an auxiliary explanatory item are extracted for each of a plurality of diseases. Therefore, the output control function 124 may display, as content, a directed graph in which a main explanatory item and an auxiliary explanatory item are associated with each disease.

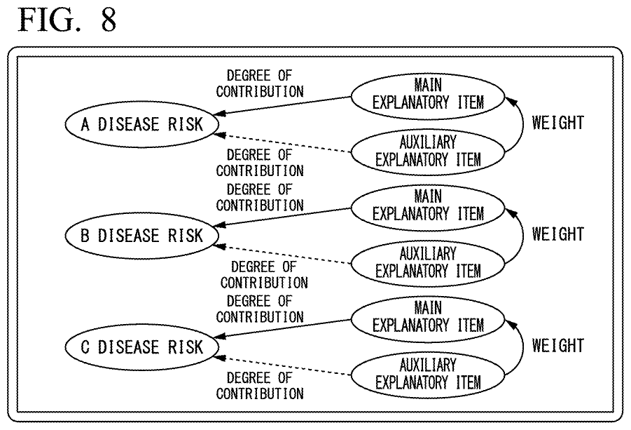

FIG. 8 is a diagram showing another example of content displayed on the display 113a. As illustrated, when a plurality of disease risks for disease A, disease B, and disease C are calculated, and a main explanatory item and an auxiliary explanatory item are extracted for each disease, the output control function 124 displays, as content, three directed graphs in which nodes of main explanatory items and auxiliary explanatory items are associated with the respective nodes of disease A, disease B, and disease C.

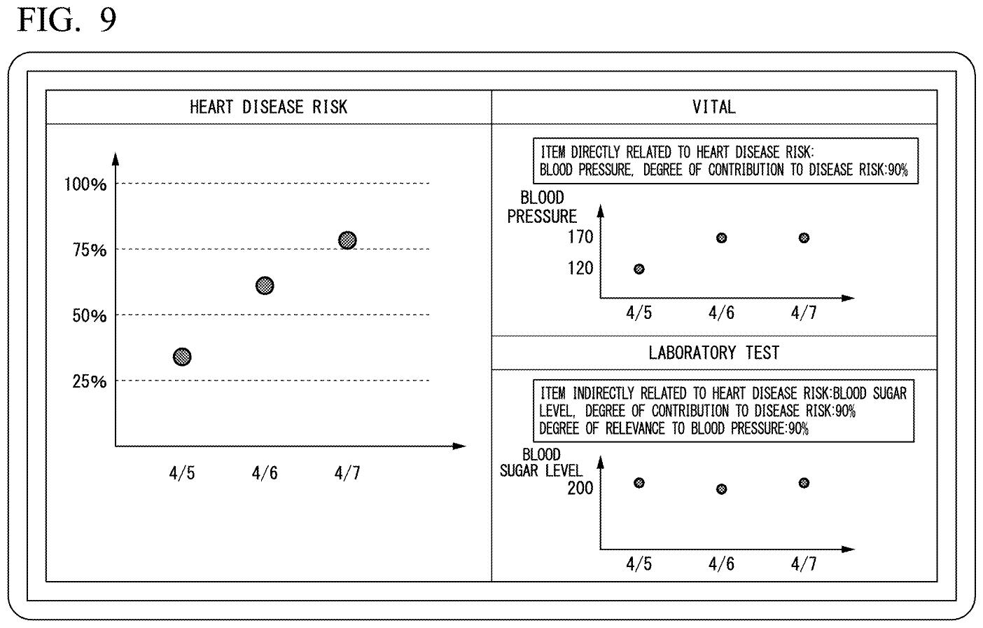

FIG. 9 is a diagram showing another example of content displayed on the display 113a. For example, it is conceivable that a disease risk changes or medical treatment information extracted as a main explanatory item or an auxiliary explanatory item changes over time. In such a case, the output control function 124 may display a disease risk and an explanatory item that change over time as graphs.

According to the embodiment described above, the processing circuitry 120 of the medical information processing device 100 extracts medical treatment information related to diagnostic support information as a main explanatory item (an example of “first medical treatment information”) from a plurality of pieces of medical treatment information input to the machine learning model 200 on the basis of a medical relevance database (an example of a “corresponding relationship”) in which the presence or absence of medical relevance of a plurality of pieces of medical treatment information such as a blood pressure and a heart rate is associated with the diagnostic support information, and a degree of contribution of each of a plurality of pieces of medical treatment information output by the machine learning model 200 to the diagnostic support information. Furthermore, the processing circuitry 120 extracts medical treatment information related to the main explanatory item as an auxiliary explanatory item (an example of “second medical treatment information”) from the plurality of pieces of medical treatment information input to the machine learning model 200 on the basis of a degree of contribution of each of the plurality of pieces of medical treatment information output by the machine learning model 200 to the diagnostic support information and weights (degrees of relevance) between the plurality of pieces of medical treatment information. Then, the processing circuitry 120 causes the display 113a to display content including the diagnostic support information, the main explanatory item, and the auxiliary explanatory item or transmits the content to an external device via the communication interface 111.

In this manner, relevance between “diagnostic support information” and “medical treatment information whose medical relevance is unknown (that is, auxiliary explanatory item)” can be visualized and presented to a user when medical treatment information whose medical relevance to the diagnostic support information is unknown has been extracted as an auxiliary explanatory item as the grounds for the diagnostic support information (disease risk) output by the machine learning model 200. As a result, the user can visually understand the relevance between the “diagnostic support information” and the “medical treatment information whose medical relevance is unknown,” and thus can investigate the cause of the disease and obtain new medical knowledge.

### Other Embodiments

Hereinafter, other embodiments (modified examples of the above-described embodiment) will be described. Although the extraction function 123 extracts, as candidates for auxiliary explanatory items, medical treatment information having no medical relevance to diagnostic support information (medical treatment information to which “0” is allocated in the medical relevance database of FIG. 4) from other pieces of medical treatment information for which weights with respect to main explanatory items have been calculated in the above-described embodiment, the present invention is not limited thereto.

For example, the extraction function 123 may extract, as candidates for auxiliary explanatory items, medical treatment information that is not associated with the presence or absence of medical relevance to diagnostic support information in the medical relevance database and is not present in the database from other pieces of medical treatment information for which weights with respect to main explanatory items have been calculated. That is, the extraction function 123 may extract, as candidates for auxiliary explanatory items, medical treatment information to which “1” or “0” is not allocated and which does not even have a field on the medical relevance database. The machine learning model 200 described above is a model with versatility.

Therefore, even if medical treatment information which is not registered in the medical relevance database and for which the presence or absence of relevance is unknown is input to the machine learning model 200, the machine learning model 200 can calculate diagnostic support information, a degree of contribution of medical treatment information, and weights (degrees of relevance) between a plurality of pieces of medical treatment information. In this manner, even medical treatment information whose medical relevance is unknown and which is not present in the medical relevance database (medical treatment information whose medical relevance is unknown that could not have been assumed at the time of creating the medical relevance database) can be extracted as candidates for auxiliary explanatory items, and thus the user can further understand output results of the machine learning model.

Further, although diagnostic support information is information representing, as a quantitative risk, a probability of a patient having already suffered from a target disease (cardiac disease, cancer, or the like) at the time of medical treatment or a probability of the patent suffering from the target disease in a certain time in the future in the above-described embodiment, the present invention is not limited thereto. For example, the diagnostic support information may quantitatively represent risks related to other medical factors or medical events that are different from diseases, such as a survival rate, a mortality rate, a readmission rate, a health level, severity, and an adverse event risk.

Although several embodiments have been described, these embodiments are presented as examples and are not intended to limit the scope of the invention. These embodiments can be implemented in various other forms, and various omissions, substitutions, and modifications can be made without departing from the scope of the invention. These embodiments and their modifications are included in the scope and spirit of the invention, as well as the scope of the invention described in the claims and equivalents thereof.

What is claimed is:

1. A medical information processing device for supporting interpretation of output results of a model that outputs diagnostic support information which is information for supporting diagnosis of a subject in response to input of a plurality of pieces of medical treatment information obtained at the time of medical treatment of the subject, the medical information processing device comprising a processing circuitry configured to:
extract first medical treatment information related to the diagnostic support information from among the plurality of pieces of medical treatment information on the basis of a corresponding relationship in which the diagnostic support information and the plurality of pieces of medical treatment information are associated, and a degree of contribution of each of the plurality of pieces of medical treatment information input to the model at the time of causing the model to output the diagnostic support information to the diagnostic support information;
extract second medical treatment information related to the first medical treatment information from among the plurality of pieces of medical treatment information on the basis of degrees of mutual relevance of the medical treatment information input to the model at the time of causing the model to output the diagnostic support information and the degree of contribution of each of the plurality of pieces of medical treatment information; and
cause a display unit to display the diagnostic support information, the first medical treatment information, and the second medical treatment information.

2. The medical information processing device according to claim 1, wherein the corresponding relationship is information in which the presence or absence of medical relevance of each of the plurality of pieces of medical treatment information is associated with the diagnostic support information.

3. The medical information processing device according to claim 2, wherein the processing circuitry extracts, as the first medical treatment information, a predetermined number of pieces of high-ranking medical treatment information having high degrees of contribution from among the plurality of pieces of medical treatment information having medical relevance to the diagnostic support information in the corresponding relationship.

4. The medical information processing device according to claim 2, wherein the processing circuitry extracts, as the first medical treatment information, medical treatment information having a degree of contribution equal to or greater than a threshold value from among the plurality of pieces of medical treatment information having medical relevance to the diagnostic support information in the corresponding relationship.

5. The medical information processing device according to claim 2, wherein the processing circuitry extracts, as the second medical treatment information, medical treatment information having no medical relevance to the diagnostic support information in the corresponding relationship or medical treatment information for which the presence or absence of medical relevance is not associated with the diagnostic support information in the corresponding relationship and which is not present in the corresponding relationship.

6. The medical information processing device according to claim 5, wherein the processing circuitry extracts, as the second medical treatment information, a predetermined number of pieces of high-ranking medical treatment information having high degrees of relevance to the first medical treatment information from among medical treatment information which has no medical relevance to the diagnostic support information or for which the presence or absence of medical relevance is not associated with the diagnostic support information.

7. The medical information processing device according to claim 5, wherein the processing circuitry extracts, as the second medical treatment information, medical treatment information whose degree of relevance to the first medical treatment information is equal to or greater than a threshold value from among the medical treatment information which has no medical relevance to the diagnostic support information or for which the presence or absence of medical relevance is not associated with the diagnostic support information.

8. The medical information processing device according to claim 1, wherein the processing circuitry further extracts third medical treatment information related to the second medical treatment information from among the plurality of pieces of medical treatment information on the basis of the degrees of mutual relevance of the medical treatment information, and the degree of contribution of each of the plurality of pieces of medical treatment information.

9. The medical information processing device according to claim 1, wherein the processing circuitry determines a display mode at the time of causing the display unit to display the diagnostic support information, the first medical treatment information, and the second medical treatment information on the basis of the degree of contribution of the first medical treatment information to the diagnostic support information, the degree of contribution of the second medical treatment information to the diagnostic support information, and the degree of mutual relevance of the first medical treatment information and the second medical treatment information.

10. The medical information processing device according to claim 1, wherein the model further outputs the degree of contribution of each of the plurality of pieces of medical treatment information and the degree of mutual relevance of the medical treatment information in response to input of the plurality of pieces of medical treatment information.

11. A medical information processing method using a medical information processing device for supporting interpretation of output results of a model that outputs diagnostic support information which is information for supporting diagnosis of a subject in response to input of a plurality of pieces of medical treatment information obtained at the time of medical treatment of the subject, the medical information processing method comprising:
extracting first medical treatment information related to the diagnostic support information from among the plurality of pieces of medical treatment information on the basis of a corresponding relationship in which the diagnostic support information and the plurality of pieces of medical treatment information are associated, and a degree of contribution of each of the plurality of pieces of medical treatment information input to the model at the time of causing the model to output the diagnostic support information to the diagnostic support information;
extracting second medical treatment information related to the first medical treatment information from among the plurality of pieces of medical treatment information on the basis of degrees of mutual relevance of the medical treatment information input to the model at the time of causing the model to output the diagnostic support information and the degree of contribution of each of the plurality of pieces of medical treatment information; and
causing a display unit to display the diagnostic support information, the first medical treatment information, and the second medical treatment information.

12. A computer-readable non-transient storage medium storing a program executed by a computer for supporting interpretation of output results of a model that outputs diagnostic support information which is information for supporting diagnosis of a subject in response to input of a plurality of pieces of medical treatment information obtained at the time of medical treatment of the subject, the program comprising:
extracting first medical treatment information related to the diagnostic support information from among the plurality of pieces of medical treatment information on the basis of a corresponding relationship in which the diagnostic support information and the plurality of pieces of medical treatment information are associated, and a degree of contribution of each of the plurality of pieces of medical treatment information input to the model at the time of causing the model to output the diagnostic support information to the diagnostic support information;
extracting second medical treatment information related to the first medical treatment information from among the plurality of pieces of medical treatment information on the basis of degrees of mutual relevance of the medical treatment information input to the model at the time of causing the model to output the diagnostic support information and the degree of contribution of each of the plurality of pieces of medical treatment information; and
causing a display unit to display the diagnostic support information, the first medical treatment information, and the second medical treatment information.
